(19) United States
(12) Patent Application Publication
(10) Pub. No.: US 2010/0331248 A1
(43) Pub. Date: Dec. 30, 2010

(54) COMPOSITIONS AND METHODS FOR TREATING DIABETES USING LISOFYLLINE AND ISLET NEOGENESIS ASSOCIATED PEPTIDE

(75) Inventors: Mary Ann Latona Nadler, Charlottesville, VA (US); Jerry L. Nadler, Charlottesville, VA (US)

Correspondence Address: NIXON & VANDERHYE, PC, 901 NORTH GLEBE ROAD, 11TH FLOOR, ARLINGTON, VA, 22203, US

(73) Assignee: DIAKINE THERAPEUTICS, INC., Charlottesville, VA (US)

(21) Appl. No.: 12/865,027

(22) PCT Filed: Feb. 2, 2009
(86) PCT No.: PCT/US09/00661

§ 371 Date: Jul. 28, 2010

Related U.S. Application Data
(60) Provisional application No. 61/025,232, filed on Jan. 31, 2008.

(51) Int. Cl. A61K 38/22; A61K 31/522; A61P 3/10

(52) U.S. Cl. 514/6.9; 514/263.31

(57) ABSTRACT

Pharmaceutical compositions and methods are provided for treating diabetes and/or restoring β-cell mass and function in a mammal in need thereof. Type 1 diabetes mellitus (T1DM) is an autoimmune disorder characterized by immune damage to pancreatic beta-cells. Lisofylline (LSF) is an immunomodulator that reduces cytokine signaling and reduces the onset of T1DM in non-obese diabetic (NOD) mice. A combination therapy with both LSF (pretreatment) and INGAP provides protection from autoimmune destruction. The concomitant or combination of LSF and INGAP after pre-treatment with LSF is an effective therapy for a disease or condition resulting from the loss of pancreatic islet cells or insulin production in a mammal.

### FIELD OF THE INVENTION

The invention relates to the use of pharmaceutical compositions and methods for using same for (1) restoring β-cell mass and function in an individual in need thereof; (2) preventing the development of, or reversing, Type 1 diabetes mellitus (T1DM) in an individual in need thereof; (3) for preventing the development of, or reversing, latent autoimmune diabetes of adults (LADA) in an individual in need thereof; and/or (4) for treating Type 2 diabetes mellitus (T2DM) by increasing the number of functional insulin-producing cells (e.g., β-cells) in an individual in need thereof.

### BACKGROUND OF THE INVENTION

Insulin is a hormone produced in the pancreas by beta cells (β-cells). The function of insulin is to regulate the amount of glucose (sugar) in the blood, which enters cells through receptors that accept insulin and allow glucose to enter. Once inside, glucose can be used by an organism as fuel. Excess glucose is stored in the liver and muscles in a form called glycogen. When blood glucose levels are low, the liver releases glycogen to form glucose. Without insulin, glucose has difficulty entering cells, which in turn, causes myriad deletrious effects.

Since about 1922, insulin has been the only available therapy for the treatment of type diabetes and other conditions related to lack of or diminished production of insulin. Despite decades of research and the advent of pancreatic islet cell transplantation in 1974 and newer claims of success resulting from the Edmonton Protocol for islet cell transplantation, the success has not been replicated in the United States. At four years post-transplant, fewer than 10% of patients who have received islet cell transplants remain insulin independent. Additionally, despite new immune suppression protocols, there is an 18% rate per patient of serious side effects.

Diabetes (Type 1, 2 or LADA) is one of the most common metabolic diseases affecting hundreds of millions of individuals worldwide. In persons with diabetes, the pancreas produces no insulin, too little insulin to control blood sugar, or defective insulin. Without insulin, these symptoms progress to dehydration, resulting in low blood volume, increased pulse rate, and dry, flushed, skin. In addition, ketones accumulate in the blood faster than the body is able to eliminate them through the urine or exhaled breath. Respiration becomes rapid, and shallow and breath has a fruity odor. Other symptoms indicating a progression towards diabetic ketoacidotic coma (DKA) include vomiting, stomach pains, and a decreased level of consciousness. The disease leads to serious complications, including hyperglycemia, macroangiopathy, microangiopathy, neuropathy, nephropathy and retinopathy. As a result, diabetes adversely affects the quality of life.

There are two forms of diabetes mellitus: (1) insulin dependent or T1DM (a.k.a., Juvenile Diabetes, Brittle Diabetes, Insulin Dependent Diabetes Mellitus (IDDM)) and (2) non-insulin-dependent or Type II diabetes (a.k.a., NIDDM). T1DM develops most often in young people but can appear in adults. T2DM develops most often in middle aged and older adults, but can appear in young people. Diabetes is a disease believed to be derived from multiple causative factors and characterized by elevated levels of plasma glucose (hyperglycemia) in the fasting state or after administration of glucose during an oral glucose tolerance test.

T1DM is an autoimmune disease condition characterized by high blood glucose levels caused by a total lack of insulin, i.e., a complete loss of pancreatic β-cell function and mass. T1DM occurs when a person's immune system attacks the insulin producing β-cells in the pancreas and destroys them. It is believed that the Interleukin 12 (IL-12) family of cytokines and downstream activation of Signal Transducers and Activators of Transcription (STAT) family members, e.g., STAT-4, which are believed to be regulators of T cell differentiation involved in immune responses, play a major role in the processes that lead to autoimmune β-cell destruction. The pancreas then produces little or no insulin. The most common T1DM symptoms experienced include excessive thirst (polydipsia), frequent urination (polyuria), extreme hunger (polyphagia), extreme fatigue, and weight loss. These symptoms are caused by hyperglycemia and a breakdown of body fats. Persons diagnosed with T1DM typically exhibit blood sugar levels over 300 mg and ketones present in their urine. Restoration of β-cell mass and insulin production can fully reverse the diabetic state. Evidence suggests that people with long standing T1DM have β-cells that continue to form but are undesirably destroyed by continued autoimmune destruction. Therefore, pharmaceutical compositions and methods for arresting autoimmune β-cell damage would provide an effective way to restore normal β-cell mass levels and reverse or cure T1DM.

LADA is a newly recognized subset of T1DM and is thought to account for up to 10%-20% of all cases of diabetes. LADA is often present in people initially diagnosed with T2DM. Although it has characteristics similar to adult onset T1DM, the beta-cell destruction is considered to be less aggressive in its progression.

T2DM results from a combination of insulin resistance and impaired insulin secretion but ultimately many people with T2DM show markedly reduced pancreatic β-cell mass and function which, in turn, causes Type 2 diabetic persons to have a “relative” deficiency of insulin because pancreatic β-cells are producing some insulin, but the insulin is either too little or isn't working properly to adequately allow glucose into cells to produce energy. Recent autopsy studies have shown clear evidence of ongoing β-cell death (apoptosis) in people with T2DM. Therefore, therapeutic approaches to arrest β-cell death could provide a significant treatment for reversing or curing T2DM.

Uncontrolled T2DM leads to excess glucose in the blood, resulting in hyperglycemia, or high blood sugar. A person with T2DM experiences fatigue, increased thirst, frequent urination, dry, itchy skin, blurred vision, slow healing cuts or sores, more infections than usual, numbness and tingling in feet. Without treatment, a person with T2DM will become dehydrated and develop a dangerously low blood volume. If T2DM remains uncontrolled for a long period of time, more serious symptoms may result, including severe hyperglycemia (blood sugar over 600 mg) lethargy, confusion, shock, and ultimately “hyperosmolar hyperglycemic non-ketotic coma.” Persistent or uncontrolled hyperglycemia is associated with increased and premature morbidity and mortality. As such, therapeutic control of glucose homeostasis, lipid metabolism, obesity, and hypertension are critically important in the clinical management and treatment of diabetes mellitus.

The object of diabetes treatments is to prevent the occurrence of the above-mentioned chronic complications, slow disease progression by improving hyperglycemic status, or reversing/curing it. Conventional methods for treating diabetes have included administration of fluids and insulin in the case of Type I diabetes and administration of various hypoglycemic agents in the case of Type II diabetes. Hypoglycemic agents such as insulin preparations, insulin secretagogues, insulin sensitizers and α-glucosidase inhibitors have been widely applied as the method for the clinical treatment. Examples include acarbose (PrecoseJ), glimeprimide (AmarylJ), metformin (Glucophage7), nateglinide (Starlix7), pioglitazone (Actos7), repaglinide (PrandinJ), rosiglitazone (Avandia7), sulfonylureas, Orlistat (Xenical7), exenatide (Byetta), and the like. Many of the known hypoglycemic agents, however, exhibit undesirable side effects and are toxic in certain cases. For example, in the case of the diabetic patients with seriously lowered pancreatic insulin secretion, effectiveness of insulin secretagogues and insulin sensitizers is diminished. Similarly, in the case of the diabetic patients whose insulin resistance is significantly high, effectiveness of insulin preparations and insulin secretagogues is diminished.

In principle, diabetes mellitus could be “cured” by a successful transplant of the tissue containing cells that secrete or produce insulin, i.e., the islets of Langerhans. Transplantation of insulin producing cells (a.k.a., islets) has been tried as a method to reverse or cure T1DM, but there are significant risks associated with the surgery and with the toxic immunosuppression type drugs that need to be taken to prevent or mitigate allograft rejection and autoimmune reoccurrence. Immunosuppression drugs act by reducing the activity of a recipient's immune system so that the transplanted insulin producing cells are not rejected. Such immunosuppression, however, entails substantial risks and there are considerable difficulties attendant in minimizing the antigenic differences (matching) between a donor and a recipient that increases the costs and reduces the availability of this mode of therapy. In addition, conventional immunosuppression is generally not successful in enabling islet transplantation. Moreover, there are over 1 million people with T1DM in the United States today, but the supply of cadaveric pancreatic tissue for islets is limited. For instance, only 6,000 organs are available per year and 2 or 3 organs are needed to provide enough islets to reverse T1DM in one person. Therefore, providing a new source of functioning (insulin producing) β-cells is urgently needed. In addition, if a diabetic patient's own cells (pancreatic or other cell types) could be genetically engineered or induced to grow and differentiate into functioning β-cells, then there would be little or no need to use toxic anti-rejection medications. As previously mentioned, there continues to be the capacity for new β-cell formation in people with T1DM. However, continued autoimmunity leads to active destruction of any newly formed or transplanted β-cells. Development of new immunomodulating agents would provide a new way to fully reverse β-cell disfunction in T1DM without the need for islet cell transplantation or toxic anti-rejection immunosuppressants. Further, the pre-treatment followed by combination therapy approach provided by the invention would be a major improvement in cellular replacement therapy by reducing the amount of transplanted cells needed to reverse or cure T1DM, facilitating the increase viability and growth of insulin producing cells, thereby improving success rates.

Unfortunately, insulin therapy does not treat the underlying mechanisms disease resulting in T1DM and other such conditions in which there is diminished endogenous insulin production. The therapies, methods, modalities, and treatments described herein are the first to address the many facets of the cause and complications of diabetes. The unique therapies provided by the invention encompass diverse aspects diabetology, metabolism, and immunology. These therapies include those that bring the many different hormones, in addition to insulin, that are diminished or absent in T1DM. The methods of the invention provide for the regeneration of new insulin producing cells and immuno-modulation that together serve to ameliorate, diminish, or abolish the need for insulin among patients with T1DM and other conditions associated with inadequate insulin production and secretion.

### Islet Neogenesis Associated Protein (INGAP)

One islet stimulating hormone is Islet Neogenesis Associated Peptide (“INGAP” or “INGAP peptide”). INGAP (including analogs and derivatives thereof) is a member of the Reg3 family of pancreatic proteins and can induce new islet formation and restore euglycemia in streptozotocin-induced diabetic mice. INGAP is a 15 amino acid peptide with the following sequence: Ile-Gly-Leu-His-Asp-Pro-Ser-His-Gly-Thr-Leu-Pro-Asn-Gly-Ser (MW 1501.4). INGAP peptide is the active core of a 168 kDa protein which has demonstrated islet neogenesis activity in several species. INGAP peptide causes the growth of new, fully-functioning islets from progenitor cells. To date, the peptide has been shown to increase β-cell mass and insulin production both in various animal models and in cultures of human tissue. It has also been shown to be safe in clinical studies and an open Investigational New Drug Application (IND) is on file with the United States Food and Drug Administration (FDA). However, because T1DM is associated with loss of β-cell mass resulting from an autoimmune response, administration of INGAP peptide alone does not appear to be a optimally suitable long-term solution because autoimmune mechanisms that destroy original β-cells would also hinder or preclude the buildup of sufficient β-cell mass under a regimen involving INGAP peptide as a monotherapy. Suitable preparations of INGAP may be obtained in accordance with, inter alia, the teachings in U.S. Reissue Pat. Nos. 39299, 39351 and 39062, the entire disclosures of which are incorporated herein by reference.

Lisofylline (“LSF”), a synthetic, modified methylxanthine, has been shown to prevent autoimmune diabetes, partially due to its anti-inflammatory function by reducing inflammatory cytokine production, including tissue necrosis factor-α (TNF-α,) interferon-γ (IFN-γ), and interleukin-1β (IL-1β). LSF effectively suppresses T cell activation and differentiation via inhibition of the signal transducer and activator of transcription-4 (STAT4)-mediated interleukin-12 (IL-12) signaling, which can prevent autoimmune diabetes and protect transplanted islets from autoimmune destruction. LSF also has anti-inflammatory actions, contributing to preservation of islet viability and function. LSF maintains beta-cell insulin secretory function in the presence of inflammatory cytokine insult and regulates immune cellular function to suppress autoimmunity. LSF also enhances insulin secretion in isolated islets and in transformed beta-cell lines in response to glucose stimulation in vitro and prevents the onset of autoimmune diabetes. LSF alone did not reverse autoimmune diabetes in non-obese diabetic (NOD) mice when hyperglycemia became detectable, however, LSF alone was able to stabilize but not normalize blood glucose levels in some cases as long as the treatment was continued. This suggests that LSF alone does not appear to be capable of inducing remission of diabetes. LSF has also been shown to be safe in clinical studies and an open IND is also on file with the FDA.

To date, there has been no single/combination or concomitant therapy or treatment protocol that has been successfully used to treat the underlying disease mechanisms of T1DM or conditions in which there is a lack of or diminished insulin production. There remains a need for new methods and pharmaceutical compositions for treating T1DM mellitus. Especially needed are methods and compositions that can also treat the many other conditions in which the lack of or diminished insulin production has a causative role or contributes to the symptoms of patients in need of treatment. At present, there appears to be no treatment that ameliorates the symptoms of T1DM by targeting the underlying disease mechanism. There also remains a need for more effective pharmaceutical compositions and methods that utilize immunomodulating agents as a pretreatment followed by the same or similar immunomodulating agents in combination with a β-cell growth and/or differentiating factor to restore normal β-cell mass and/or function in subjects suffering from diabetes.

### SUMMARY OF THE INVENTION

Now it has been surprisingly found that pre-treatment with LSF followed by treatment with a combination of LSF and INGAP or INGAP alone is useful for restoring normal β-cell mass and/or function; preventing the development of, or reversing, T1DM, Latent Autoimmune Diabetes of Adults (LADA), and/or T2DM; and increasing the number of functional insulin producing cells in an individual in need thereof as compared with previous pharmaceutical compositions and methods of treatment. In one aspect, the invention provides for the use of compounds or agents that can block cytokine signaling or formation and thereby prevent autoimmune damage to regenerated/emerging new insulin producing cells. Without using an agent to block the autoimmune process, β-cell differentiation and/or growth promoting agents will not be clinically effective because simultaneous regeneration of β-cells and prevention of autoimmune reactions would not be realized. The success of pre-treatment step described herein is wholly unexpected.

In an exemplary embodiment, the invention provides pharmaceutical compositions that may be used in a method for the prevention and treatment (including reversal and cure) of mammals (including humans and animals) suffering from diseases or conditions caused by, or associated with, diabetes mellitus (Type 1, LADA and Type 2), hyperglycemia, dyslipidemia, hyperlipidemia, hypercholesterolemia, hypertriglyceridemia, hyperinsulinemia, diabetic complications, glucose intolerance, obesity or the like.

An exemplary method of the invention comprises:

(1) administering to a mammal in need thereof, e.g., a human patient or animal, a pharmaceutically or therapeutically effective amount of LSF followed by;

(2) the administration of a preventative- or therapeutically-effective amount of a pharmaceutical composition comprising, in admixture with a pharmaceutically acceptable carrier, diluent, excipient, adjuvant or vehicle: (a) LSF and (b) INGAP, either alone or in admixture with a diluent or in the form of a medicament.

It is believed that pre-treatment with LSF to inhibit IL-12 overproduction, or to inhibit the production of cytokines such as IL-23 and IL-27 which promote STAT-4 activation and autoimmune disorders such as T1DM and LADA development is critical. Overproduction of inflammatory cytokines such as IL-6, IL-1, beta interferon gamma, TNF-α, etc. and the resultant excessive Th1 type responses can be suppressed by modulating IL-12, IL-23 and/or IL-27 production. Therefore, LSF that down-regulate IL-12, IL-23 and/or IL-27 production are used as a pre-treatment to “quiet” the immune system without the deleterious side effects experienced with immunosuppressants. After pre-treatment, various combination treatments, including those disclosed in U.S. Pat. No. 7,393,919 or U.S. Patent Application No. 2006-0198839, may be employed.

Exemplary biological/immune response modifying (immunomodulating) or anti-inflammatory compounds or agents include, without limitation, members of the group consisting of: LSF described in W0/00/61583 (corresponding to U.S. Pat. No. 6,774,130 (the entire disclosure of which is incorporated herein by reference) or any other small molecule or peptide or method capable of blocking interleukin 12, interleukin 23 or activation and/or expression of STAT-4, as further described below.

Lisofylline (a.k.a. 1-(5-R-hydroxyhexyl)-3,7-dimethylxanthine) is a synthetic, modified xanthine based compound have the following structural formula:

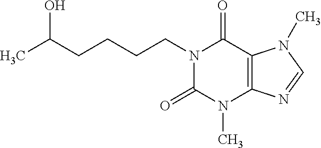

Without wishing to be bound by any theory of operation or mode of action, LSF exhibits anti-inflammatory function by reducing inflammatory cytokine production or downstream effects (including, without limitation, IL-12, IL-23, IL-27, TNF-α, IFN-γ, IL-6 and IL-1β), selectively suppressing neutrophil and leukocyte adhesion and phagocytic activity, and decreasing neutrophil migration and degranulation during sepsis. More significantly, LSF allows retention of beta-cell insulin secretory function after inflammatory cytokine insult and regulates immune cellular function to prevent autoimmunity. In addition, LSF also exhibits the ability to ameliorate hemorrhage-induced tissue injury and to preserve tissue function during decreased blood flow or in poorly ventilated conditions. LSF also inhibits phosphatidic acid formation to prevent oxidant-mediated capillary leak, thus reducing capillary barrier damage caused by oxidative stress. All of these characteristics render LSF capable of improving the clinical outcome by their use as a pre-treatment prior to administration of mono or combination therapies.

The pharmaceutical compositions useful in the invention may conveniently be provided, or is otherwise envisioned in the form of formulations suitable for parenteral (including intravenous, intramuscular and subcutaneous) nasal, oral administration or pulmonary via a inhalation device. In some cases, it will be convenient to provide a biological/immune response modifier or anti-inflammatory agent, as described herein, and any compound or agent (small molecule or peptide) that facilitates growth and/or differentiation of pancreatic β-cells or any insulin producing cell, each in a single composition or solution for administration together. A suitable administration format may best be determined by a medical practitioner for each patient individually. Suitable pharmaceutically acceptable carriers and their formulation are described in standard formulation treatises, e.g., Remington's Pharmaceutial Sciences by E. W. Martin. See also Wang, Y. J. and Hanson, M. A. “Parenteral Formulations of Proteins and Peptides: Stability and Stabilizers,” Journal of Parenteral Science and Technology, Technical Report No. 10, Supp. 42:2S (1998).

Accordingly, the invention provides use of the pharmaceutical compositions and agents described herein in conjunction with (1) methods for restoring β-cell mass and function in an individual in need thereof; (2) methods for preventing the development of, or reversing, T1DM in an individual in need thereof; (3) methods for preventing the development of, or reversing, latent autoimmune diabetes of adults (LADA) in an individual in need thereof; and (4) methods for treating T2DM by increasing the number of functional insulin producing cells (e.g., β-cells) in an individual in need thereof.

The above compounds and agents used in the pharmaceutical composition of the invention may be purchased from conventional sources, may be readily isolated from and purified (isolated) from natural sources or may be synthesized using conventional techniques known to the skilled artisan using readily available starting materials.

Other technical features and advantages of the invention will be set forth, in part, in the description that follows, or may be learned from practicing or using the invention. The advantages of the invention may be realized and attained by means of technical features described below and pointed out in the appended claims. It is to be understood that the foregoing general description and the following detailed description are merely exemplary and explanatory and should not to be viewed as being restrictive of the invention, as claimed.

### BRIEF DESCRIPTION OF THE DRAWINGS

The accompanying drawings, which are incorporated in, and constitute a part of the specification, illustrate or exemplify embodiments of the invention and, together with the description, serve to explain the principles and features of the invention.

FIG. 1 depicts a therapy regimen of the invention involving INGAP peptide and LSF for building and preserving β-cell mass and increasing insulin production in a mammal.

FIG. 2 show the treatment timelines for the exemplified regimens of INGAP and LSF treatments.

FIG. 3 shows the blood glucose levels (non-fasting) at the end of treatments the treatment regimen.

FIG. 4 shows the amount of insulin required during the treatment regimen period

FIG. 5 shows the remission rates of diabetes in the 5 tested groups.

FIG. 6 shows a slide (50396) from a mouse with placebo treatment.

FIG. 7 shows the improvement observed with the LSF-INGAP concurrent treatment.

FIG. 8 shows insulin staining and expression of the PDX-1.

Each of FIGS. 9 through 14 show results of Hoechst staining (blue) shows DNA, insulin (green) shows insulin, and Ki 67 (red) and further shows observed cell cycling or cell proliferation.

FIG. 15 shows insulin production of treated animals.

### DESCRIPTION OF EXEMPLARY EMBODIMENTS

All patents, patent applications and literatures cited or referenced in this description are incorporated herein by reference in their entirety. In the case of inconsistencies, the present disclosure, including definitions, will control.

The pharmaceutical compositions and methods of the invention comprise the pre-treatment with a biological/immune response modifier or anti-inflammatory agent (e.g., small molecule, antibody, peptide or gene therapy reagent) that effectively blocks comprising a biological response modifier and a β-cell growth factor in admixture with a pharmaceutically acceptable carrier, adjuvant or vehicle, wherein the pharmaceutical composition blocks or prevents the autoimmune response in a mammal by inhibiting the activity or expression of cytokines such as interleukins 12, 23 or 27, or members of the family of Signal Transducers and Activators of Transcription (STAT), suitably STAT-4, which are believed to be regulators of T cell differentiation involved in immune responses, followed by (2) administration of a combination of (a) the same or different biological/immune response modifier or anti-inflammatory agent, as described above and (b) any compound or agent (small molecule or peptide) that induces growth and/or differentiation of pancreatic β-cells or any insulin producing cell.

In another exemplary embodiment, the invention involves the use of an inventive pharmaceutical composition comprising LSF and INGAP to create or grow insulin producing cells in a test tube to be transplanted in patients by any acceptable procedure to prevent, treat or reverse T1DM, LADA or T2DM. In addition, this combined therapeutic approach can be given to a human to restore beta (insulin producing) cells in the body to prevent, treat or reverse T1DM, LADA or T2DM. Accordingly, in another exemplary embodiment, the invention provides a method for improving the outcome or success of cellular (islet cell, isolated β-cells, genetically engineered or induced (e.g., via transcription factors) β-cells) transplantation in a mammal to reverse T1DM, LADA and T2DM, comprising administering to the mammal (or cells to be transplanted) an effective amount of a pharmaceutical composition of the invention.

### Definitions

The following definitions are provided to assist the reader. Unless otherwise defined, all terms of art, notations and other scientific or medical terms or terminology used herein are intended to have the meanings commonly understood by those of skill in the art (e.g., biological, chemical, medical, etc.). In some cases, terms with commonly understood meanings are defined herein for clarity and/or for ready reference, and the inclusion of such definitions herein should not necessarily be construed to represent a substantial difference over the definition of the term as generally understood in the art.

“Optional” or “optionally” means that the subsequently described event or circumstance may or may not occur, and that the description includes, without limitation, instances where said event or circumstance occurs and instances in which it does not. For example, optionally substituted alkyl means that alkyl may or may not be substituted by those groups enumerated in the definition of substituted alkyl.

“Pharmaceutically acceptable derivative” or “prodrug” means any pharmaceutically acceptable salt, ester, salt of an ester, or other derivative of LSF which, upon administration to a recipient, is capable of providing (directly or indirectly) a compound of this invention. The term “prodrug”, as employed herein, denotes a compound that is a drug precursor which, upon administration to a subject, undergoes chemical conversion by metabolic or chemical processes to yield a pharmaceutically active compound. Particularly favored derivatives and prodrugs are those that increase the bioavailability of the compounds of this invention when such compounds are administered to a mammal (e.g., by allowing an orally administered compound to be more readily absorbed into the blood) or that enhance delivery of the parent compound to a biological compartment (e.g., the brain or lymphatic system) relative to the parent species. Prodrugs are considered to be any covalently bonded carriers which release LSF when such prodrug is administered to a mammalian subject. Exemplary prodrugs include, without limitation, derivatives where a group that enhances aqueous solubility or active transport through the gut membrane is appended to LSF. Prodrugs of LSF are prepared by modifying functional groups present in the compounds in such a way that the modifications are cleaved, either in routine manipulation or in vivo, to the parent compounds. Prodrugs include LSF wherein hydroxyl, amino, sulfhydryl, or carboxyl groups are bonded to any group that, when administered to a mammalian subject, cleaves to form a free hydroxyl, amino, sulfhydryl, or carboxyl group, respectively. Examples of prodrugs include, but are not limited to, acetate, formate and benzoate derivatives of alcohol and amine functional groups in the compounds of LSF. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference.

“Pharmaceutically acceptable salts” refer to derivatives of the disclosed compounds wherein LSF is modified by making acid or base salts of the compound of LSF. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of LSF include the conventional nontoxic salts or the quaternary ammonium salts of the compounds of LSF formed, for example, from nontoxic inorganic or organic acids. For example, such conventional non-toxic salts include, without limitation, those derived from inorganic acids such as acetic, 2-acetoxybenzoic, adipic, alginic, ascorbic, aspartic, benzoic, benzenesulfonic, bisulfic, butyric, citric, camphoric, camphorsulfonic, cyclopentanepropionic, digluconic, dodecylsulfanilic, ethane disulfonic, ethanesulfonilic, fumaric, glucoheptanoic, glutamic, glycerophosphic, glycolic, hemisulfanoic, heptanoic, hexanoic, hydrochloric, hydrobromic, hydroiodic, 2-hydroxyethanesulfonoic, hydroxymaleic, isethionic, lactic, malic, maleic, methanesulfonic, 2-naphthalenesulfonilic, nicotinic, nitric, oxalic, palmic, pamoic, pectinic, persulfanilic, phenylacetic, phosphoric, propionic, pivalic, propionate, salicylic, succinic, stearic, sulfuric, sulfamic, sulfanilic, tartaric, thiocyanic, toluenesulfonic, tosylic, undecanoatehydrochloric, and the like. The pharmaceutically acceptable salts of the invention can be synthesized from the LSF which may contain a basic or acidic moiety by conventional chemical methods, for example, by reacting the free base or acid with stoichiometric amounts of the appropriate base or acid, respectively, in water or in an organic solvent, or in a mixture of the two (nonaqueous media like ether, ethyl acetate, ethanol, isopropanol, or acetonitrile are suitable) or by reacting the free base or acid with an excess of the desired salt-forming inorganic or organic acid or base in a suitable solvent or various combinations of solvents. Lists of suitable salts are found in Remington's Pharmaceutical Sciences, 17th ed., Mack Publishing Company, Easton, Pa., 1985, p. 1418, et al., the entire disclosure of which is incorporated herein by reference.

Further, exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others. All such acid salts and base salts are intended to be pharmaceutically acceptable salts within the scope of the invention and all acid and base salts are considered equivalent to the free forms of the corresponding compounds for purposes of the invention.

“Pharmaceutically effective” or “therapeutically effective” amount of a compound of the invention is an amount that is sufficient to effect the desired therapeutic, ameliorative, palliatory, eliminatory, inhibitory or preventative effect, as defined herein, when administered to a mammal in need of such treatment. The amount will vary depending upon the subject and disease condition being treated, the weight and age of the subject, the severity of the disease condition, the manner of administration and the like, which can be readily determined by one of skill in the art. The full therapeutic effect does not necessarily occur by administration of one dose and may occur only after administration of a series of doses. Thus, a therapeutically effective amount may be administered in one or more administrations.

“Mammal” means humans and other mammalian animals.

“Treatment” refers to any treatment of a disease (e.g., any for of diabetes) or condition in a mammal, particularly a human, and includes, without limitation: (i) preventing the disease or condition from occurring in a subject which may be predisposed to the condition but has not yet been diagnosed with the condition and, accordingly, the treatment constitutes prophylactic treatment for the pathologic condition; (ii) inhibiting the disease or condition, i.e., arresting its development; (iii) relieving the disease or condition, i.e., causing regression of the disease or condition; or (iv) relieving the symptoms resulting from the disease or condition, e.g., relieving an inflammatory response without addressing the underlining disease or condition. “Treating” a condition or patient also may refer to taking steps to obtain beneficial or desired results, including clinical results. For purposes herein, beneficial or desired clinical results include, but are not limited to, alleviation or amelioration of one or more symptoms of diabetes, diminishment of extent of disease, delay or slowing of disease progression, amelioration, reduction, palliation or stabilization of the disease state, and other beneficial results described below.

As used herein, “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, a “manifestation” of a disease refers to a symptom, sign, anatomical state (e.g., lack of islet cells), physiological state (e.g., glucose level), or report (e.g., triglyceride level) characteristic of a subject with the disease.

As used herein, “reduction” of a symptom or symptoms (and grammatical equivalents of this phrase) means decreasing of the severity or frequency of the symptom(s), or elimination of the symptom(s).

As used herein, a “prophylactically effective amount” of a drug is an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose and may occur only after administration of a series of doses. Thus, a prophylactically effective amount may be administered in one or more administrations.

As used herein, “TID”, “QD” and “QHS” have their ordinary meanings of “three times a day”, “once daily,” and “once before bedtime”, respectively.

The invention also envisions the quaternization of any basic nitrogen-containing groups of the LSF compound. The basic nitrogen can be quaternized with any agents known to those of ordinary skill in the art including, without limitation, lower alkyl halides, such as methyl, ethyl, propyl and butyl chlorides, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

The dosage of active ingredient in the pharmaceutical compositions of this invention may be varied; however, it is necessary that the amount of the active ingredient be such that a suitable dosage form is obtained, which dosage can be readily determine through routine experimentation. The selected dosage depends upon the desired therapeutic effect, on the route of administration, and on the duration of the treatment. In general, an effective dosage for the activities of this invention is in the range of 1×10⁻⁷ to 200 mg/kg/day, suitably 1×10⁻⁴ to 100 mg/kg/day, which can be administered as a single dose or divided into multiple doses. Suitably the therapeutic amount is between about 0.5 mg to about 12 mg, and suitably between about 2 mg to about 8 mg, with the most exemplary dosage being between about 2 mg and about 6 mg. Unit dosage forms are acceptable.

Generally, a therapeutically effective daily dose is from about 0.001 mg to about 15 mg/kg of body weight per day of a compound of the invention; suitably, from about 0.1 mg to about 10 mg/kg of body weight per day; and still suitably, from about 0.1 mg to about 1.5 mg/kg of body weight per day. For example, for administration to a 70 kg person, the dosage range would be from about 0.07 mg to about 1050 mg per day of a compound of the invention, suitably from about 7.0 mg to about 700 mg per day, and still suitably from about 7.0 mg to about 105 mg per day. Some degree of routine dose optimization may be required to determine an optimal dosing level and pattern. Suitable dosages are well known or readily determinable by the skilled artisan. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

To practice the method of the invention, the pharmaceutical compositions of the invention can be administered by oral, parenteral (e.g., intramuscular, intraperitoneal, intravenous or subcutaneous injection, or implant), by inhalation spray, nasal, buccal, vaginal, rectal, implanted reservoir, sublingual or topical routes of administration and can be formulated with pharmaceutically acceptable carriers to provide dosage forms appropriate for each route of administration. The term “parenteral” as used herein includes subcutaneous, intracutaneous, intravenous, intramuscular, intraarticular, intraarterial, intrasynovial, intrastemal, intrathecal, intralesional and intracranial injection or infusion techniques. The invention is exemplified by any method of administering an effective amount of LSF pretreatment that effectively blocks autoimmune response or cytokine formation in a mammal, followed by treatment with INGAP (or analog thereof) alone or in combination with LSF that effectively facilitates growth and/or differentiation of pancreatic β-cells or any insulin producing cell.

Solid dosage forms for oral administration include capsules, tablets, pills, powders, pellets and granules. In such solid dosage forms, the active compound is admixed with at least one inert pharmaceutically acceptable carrier such as sucrose, lactose, or starch. Such dosage forms can also comprise, as is normal practice, additional substances other than such inert diluents, e.g., lubricating agents such as magnesium stearate. In the case of capsules, tablets and pills, the dosage forms may also comprise buffering agents. Tablets and pills can additionally be prepared with enteric coatings.

Liquid dosage forms for oral administration include pharmaceutically acceptable emulsions, solutions, suspensions, syrups, the elixirs containing inert diluents commonly used in the art, such as water. Besides such inert diluents, compositions can also include adjuvants, such as wetting agents, emulsifying and suspending agents, and sweetening, flavoring and perfuming agents.

Preparations according to this invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be dissolved in sterile water, or some other sterile injectable medium immediately before use.

Compositions for rectal or vaginal administration are suitably suppositories which may contain, in addition to the active substance, excipients such as coca butter or a suppository wax.

Compositions for nasal or sublingual administration are also prepared with standard excipients well known in the art.

The composition of the invention may include also conventional excipients of the type used in pharmaceutical compositions. For example, the composition may include pharmaceutically acceptable organic or inorganic carriers suitable for oral administration. Examples of such carriers include: sugar spheres, diluents, hydrophilic polymers, lubricants, glidants (or anti-adherents), plasticizers, binders, disintegrants, surfactants and pH modifiers.

Suitable diluents include microcrystalline cellulose, lactose, sucrose, fructose, glucose dextrose, or other sugars, dibasic calcium phosphate, calcium sulphate, cellulose, ethylcellulose, cellulose derivatives, kaolin, mannitol, lactitol, maltitol, xylitol, sorbitol, or other sugar alcohols, dry starch, dextrin, maltodextrin or other polysaccharides, inositol or mixtures thereof.

Suitable hydrophilic polymers include hydroxypropylmethyl cellulose, carbomers, polyethylene oxides, hydroxypropyl cellulose, hydroxyethyl cellulose, carboxymethylcellulose, sodium carboxymethylcellulose, carboxyvinylpolymers, polyvinyl alcohols, glucans, scleroglucans, mannans, xanthans, carboxymethylcellulose and its derivatives, methylcellulose and, in general, cellulose, crosslinked polyvinylpyrrolidone, carboxymethyl starch, potassium methacrylate-divinylbenzene copolymer, hydroxypropylcyclodextrin, alpha, beta, gamma cyclodextrin or derivatives and other dextran derivatives, natural gums, seaweed extract, plant exudate, agar, agarose, algin, sodium alginate, potassium alginate, carrageenan, kappa-carrageenan, lambda-carrageenan, fucoidan, furcellaran, laminarin, hypnea, eucheuma, gum arabic, gum ghatti, gum karaya, gum tragacanth, guar gum, locust bean gum, quince psyllium, flax seed, okra gum, arabinogalactin, pectin, scleroglucan, dextran, amylose, amylopectin, dextrin, acacia, karaya, guar, a swellable mixture of agar and carboxymethyl cellulose; a swellable composition comprising methyl cellulose mixed with a sparingly cross-linked agar; a blend of sodium alginate and locust bean gum; and the like.

Suitable glidants (or anti-adherents) include colloidal silica, fumed silicon dioxide, silica hydrogel, talc, fumed silica, gypsum, kaolin and glyceryl monostearate.

Suitable plasticizers include acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, glyceryl monocaprate.

Suitable binders include starches, hydroxypropylmethyl cellulose, hydroxypropyl cellulose, ethyl cellulose, polyvinyl pyrrolidone, acacia, guar gum, hydroxyethylcellulose, agar, calcium carrageenan, sodium alginate, gelatin, saccharides (including glucose, sucrose, dextrose and lactose), molasses, extract of Irish moss, panwar gum, ghatti gum, mucilage of isapol husk, carboxymethylcellulose, methylcellulose, veegum, larch arbolactan, polyethylene glycols, waxes and mixtures thereof.

Suitable disintegrants include starches, sodium starch glycollate, crospovidone, croscarmellose, microcrystalline cellulose, low substituted hydroxypropyl cellulose, pectins, potassium methacrylate-divinylbenzene copolymer, polyvinylalcohol, thylamide, sodium bicarbonate, sodium carbonate, starch derivatives, dextrin, beta cyclodextrin, dextrin derivatives, magnesium oxide, clays, bentonite and mixtures thereof.

Suitable surfactants include nonionic surfactants such as sorbitan sesquioleate, polyoxyethylene sorbitan monooleate, polyoxyethylene monostearate, glycerol monostearate, propylene glycol monolaurate, polyoxyethylene lauryl ether, polyoxyethylene cetyl ether or polyoxyethylene hydrogenated castor oil; and ionic surfactants such as sodium dodecyl sulfate or benzalkonium chloride; and the like.

Suitable pH modifiers include organic acids such as citric acid, fumaric acid, tartaric acid, succinic acid, ascorbic acid, acetic acid, malic acid, glutaric acid and adipic acid; salts of these acids; salts of inorganic acids and magnesium hydroxide.

In general, it has proved advantageous to administer intravenously amounts of from 0.01 mg to 10 mg/kg, suitably 0.05 to 5 mg/kg, of body weight per day and to administer orally 0.05 to 20 mg/kg, suitably 0.5 mg to 5 mg/kg of body weight per day, to achieve effective results. Nevertheless, it can at times be necessary to deviate from those dosage rates, and in particular to do so as a function of the nature and body weight of the human or animal subject to be treated, the individual reaction of this subject to the treatment, type of formulation in which the active ingredient is administered, the mode in which the administration is carried out and the point in the progress of the disease or interval at which it is to be administered. Thus, it may in some case suffice to use less than the above-mentioned minimum dosage rate, whilst other cases the upper limit mentioned must be exceeded to achieve the desired results. Where larger amounts are administered, it may be advisable to divide these into several individual administrations over the course of the day.

Article of Manufacture

In another aspect, the invention provides an article of manufacture in accordance with the invention comprises a means for holding a pharmaceutical composition(s), as previously described, suitable for administration to a patient in combination with printed labeling instructions providing a discussion of when or how a particular dosage form should be administered to the patient. The pharmaceutical composition will be contained in any suitable means or container capable of holding and dispensing the dosage form and which will not significantly interact with the composition and will further be in physical relation with the appropriate labeling advising that the dosage form exhibits an ability, or may be used, to restore β-cell mass and function in a mammal in need thereof. The labeling instructions will be consistent with the methods of treatment as described hereinbefore. For example, the labeling may be associated with a container by any means that maintain a physical proximity of the two. Further, by way of non-limiting example, they may both be contained in a packaging means such as a box or plastic shrink wrap or may be associated with the instructions being bonded to container such as with glue or adhesive that does not obscure the labeling instructions or other bonding or holding means.

The invention will be further illustrated in the following, non limiting Examples (working and prophetic). The Examples are illustrative only and do not limit the claimed invention regarding the materials, conditions, process parameters and the like recited herein.

### EXAMPLE

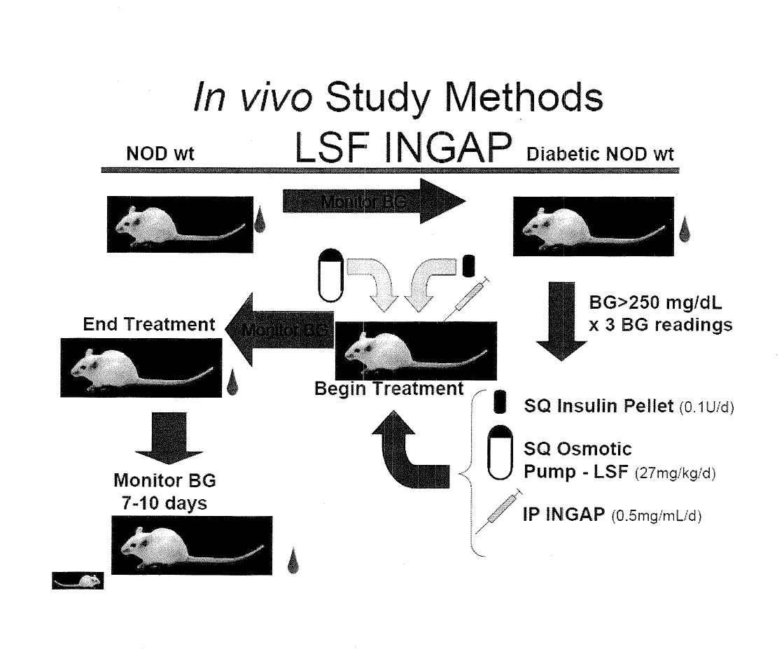

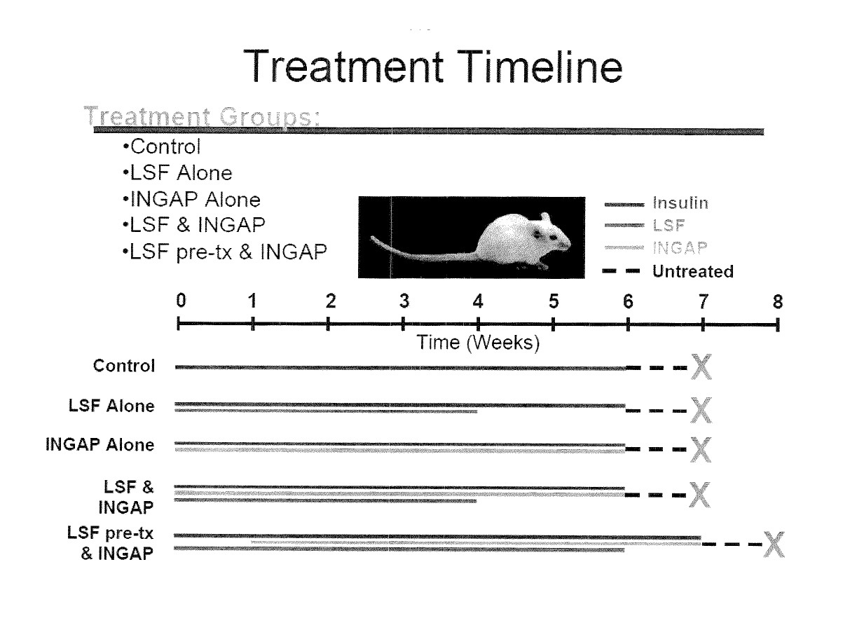

The following example illustrates a therapy regimen (FIG. 1) involving INGAP peptide and LSF for building and preserving β-cell mass and increasing insulin production in a mammal. NOD mice were monitored for diabetes by measuring blood glucose levels. Mice were allowed to develop spontaneous diabetes, which usually occurs around the age of 18 weeks. Non-fasting blood glucose levels >250 mg/dL measured for 3 different days was considered evidence of diabetes onset. Shortly after diagnosing diabetes, the NOD mice received an insulin pellet implanted subcutaneously in order to maintain them during the treatment period of 6 to 7 weeks. The pellet provides 0.1 U/d of bovine insulin. The mice were then assigned to one of 5 treatment groups:

Group 1—Normal saline via continuous subcutaneous infusion (placebo). The saline was delivered via an implantable subcutaneous osmotic mini-pump. The mice received the placebo for 4 weeks and continued on insulin for another 2 weeks. The insulin was then stopped, and the mice observed to determine if the treatment was effective.

Group 2—LSF (27 mg/kg/day via continuous subcutaneous infusion) alone. The LSF was delivered via an implantable subcutaneous osmotic mini-pump. The mice received LSF for 4 weeks, continued on insulin for another 2 weeks, and then observed to determine if the treatment was effective.

Group 3—INGAP (500 μg qd ip) alone. The mice received INGAP and insulin for 6 weeks, the treatment was then stopped, and the mice observed to determine if the treatment was effective.

Group 4—LSF (27 mg/kg/day via continuous subcutaneous infusion) plus INGAP (500 μg qd ip) administered concomitantly. The LSF was delivered via an implantable subcutaneous osmotic mini-pump. The mice received LSF for 4 weeks and INGAP and insulin for 6 weeks. The treatments were then stopped and the mice observed to determine if the treatment was effective.

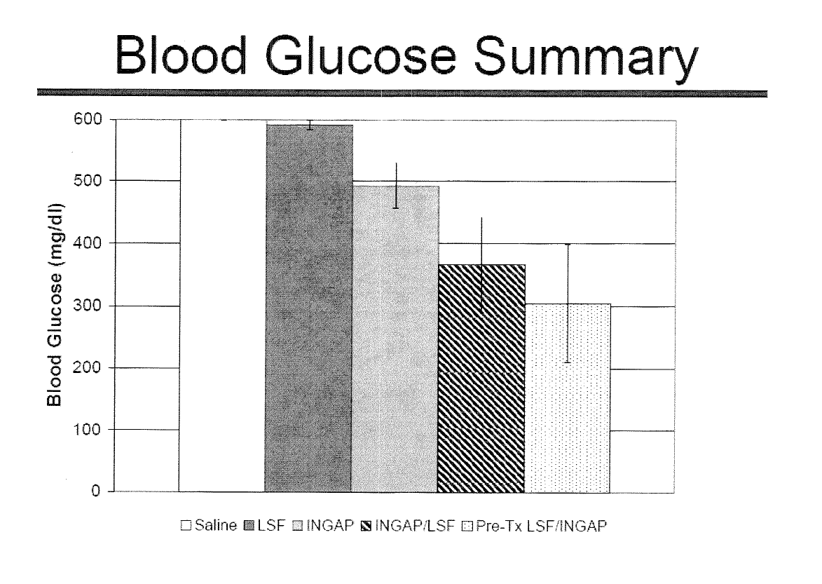

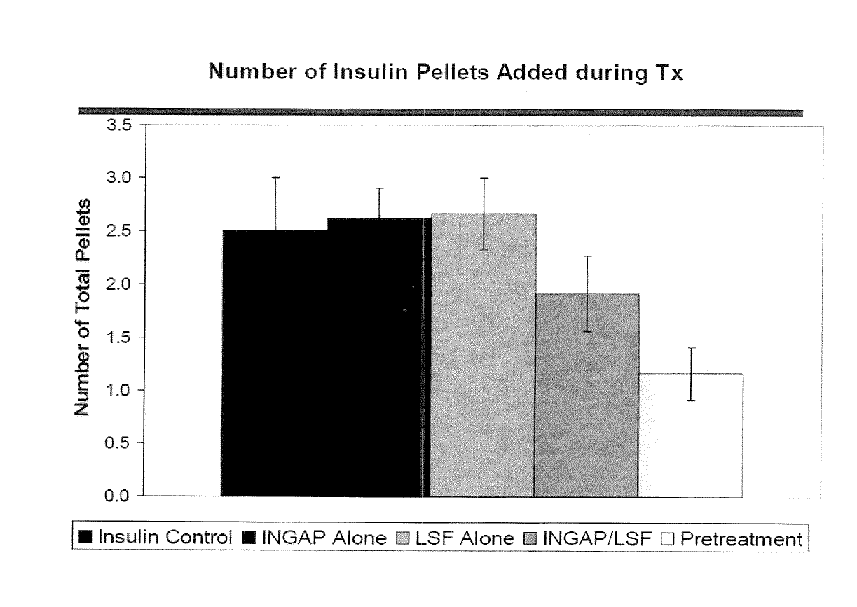

Group 5—LSF (27 mg/kg/day via continuous subcutaneous infusion) for a week of pretreatment as monotherapy. Here the immune system was first treated to ‘cool it off’ so that any new islets would be growing in a less hostile environment. INGAP was then added to the treatment regimen (at 500 μg qd ip) and administered concomitantly with the LSF for an additional 5 weeks. LSF treatment was discontinued at week 6. INGAP and Insulin were continued for 1 more week until week 7. All therapy was then discontinued and the mice observed to determine if the treatment was effective. See treatment timeline depicted in FIG. 2.

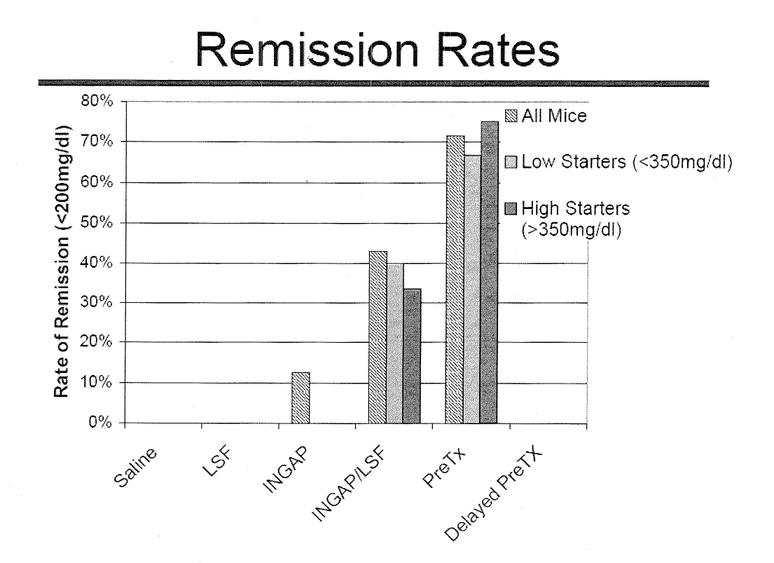

FIG. 3 shows the blood glucose levels (non-fasting) at the end of treatments. Saline, LSF alone, and INGAP alone had little or no effect on BG. Both combination treatment groups had better Blood Glucose (“BG”) levels, with the pretreatment group averaging the best non-fasting BG levels. Of note, BG levels from two mice with apparent aggressive regeneration of insulin producing cells are not included in the averages. These two mice rapidly became hypoglycemic and were sacrificed.

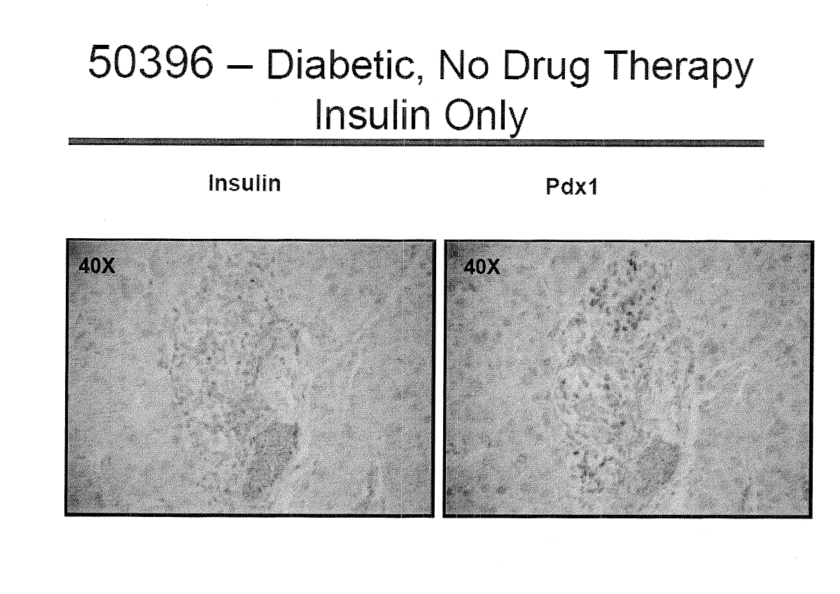

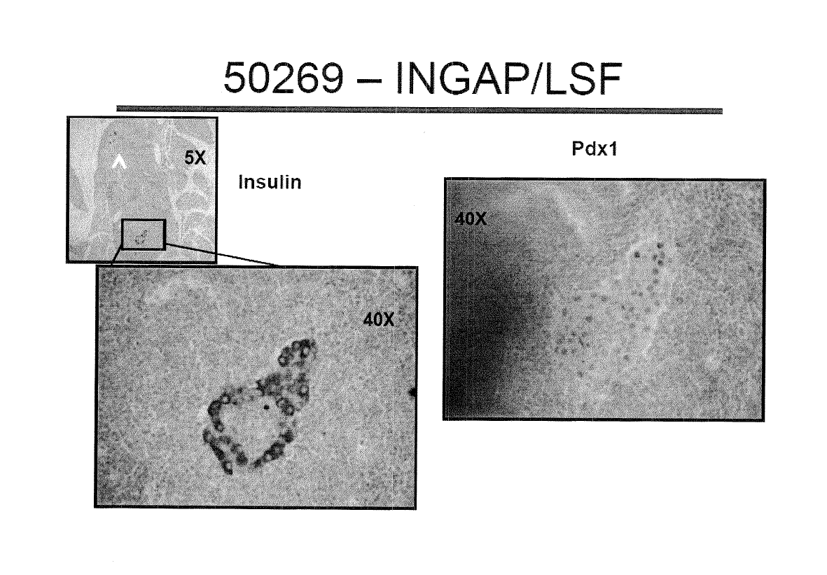

FIG. 4 shows the amount of insulin required during the treatment period for the 5 groups. The combination groups required less insulin pellets with the pretreatment group requiring on average 1.25 pellets compared to 2.5 in the placebo group.

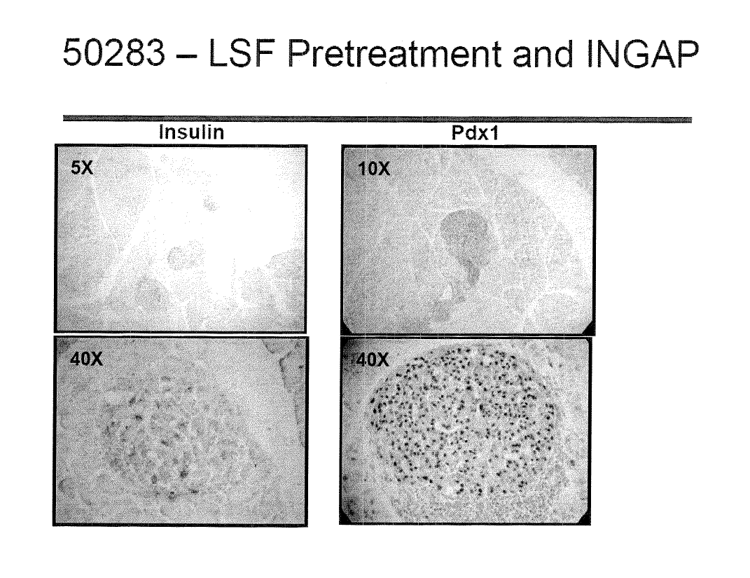

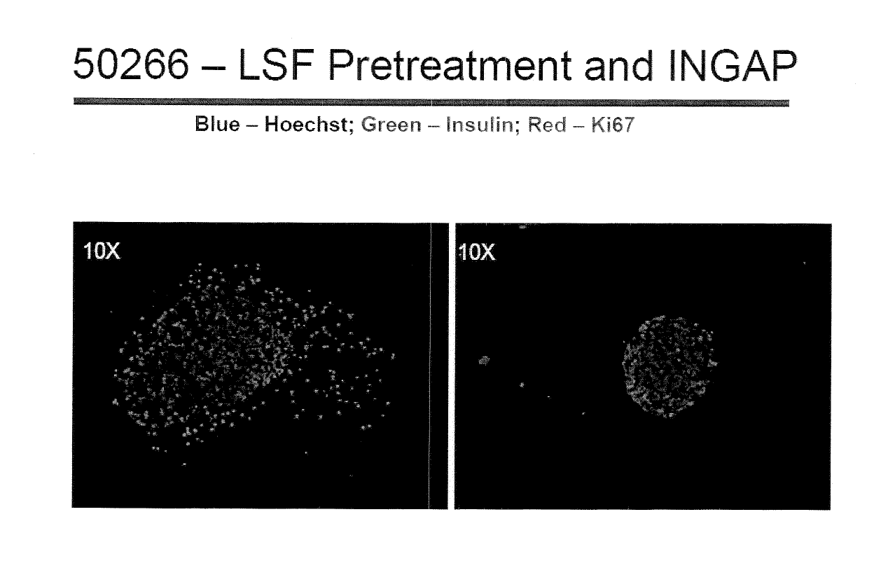

FIG. 5 shows the remission rates of diabetes in the 5 groups. No mice in the placebo or LSF alone groups remitted. Only 10% of the INGAP alone animals had a remission. Approximately 40% of the concurrently administered LSF-INGAP group remitted. Again, the pretreatment group did better with 70% responding. Of particular note is the fact that even mice with very high starting BG levels (over 350 mg/dL) responded to treatment.

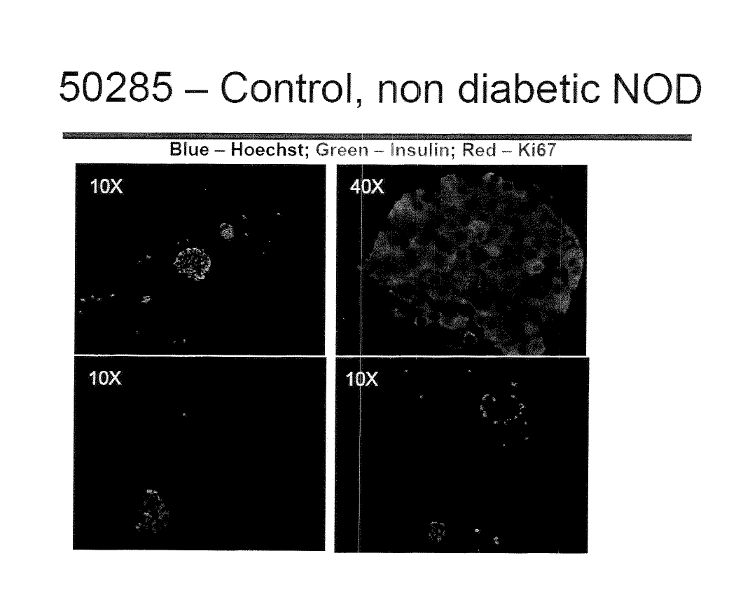

FIG. 6 shows a slide (50396) from a mouse with placebo treatment and shows no cells with insulin or of beta cell origin in the area of an islet with the transcription factor PDX 1.

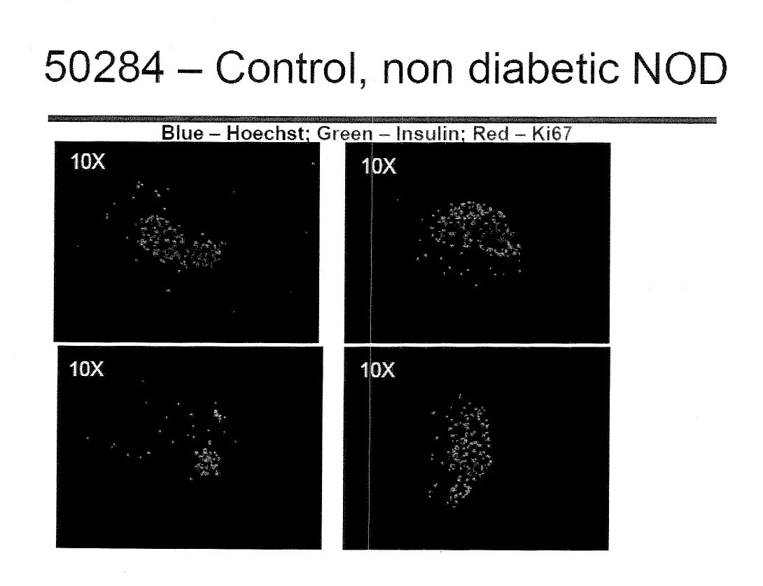

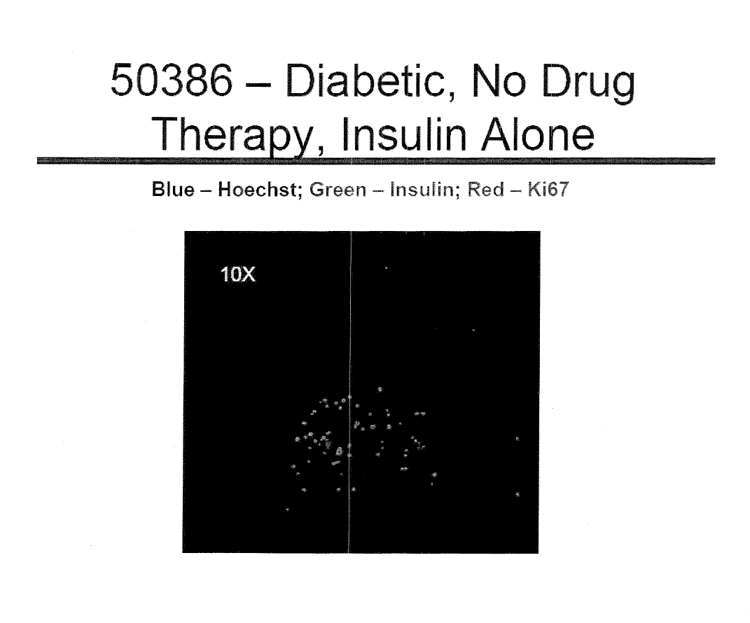

FIG. 7 shows marked improvement with the LSF-INGAP concurrent treatment.

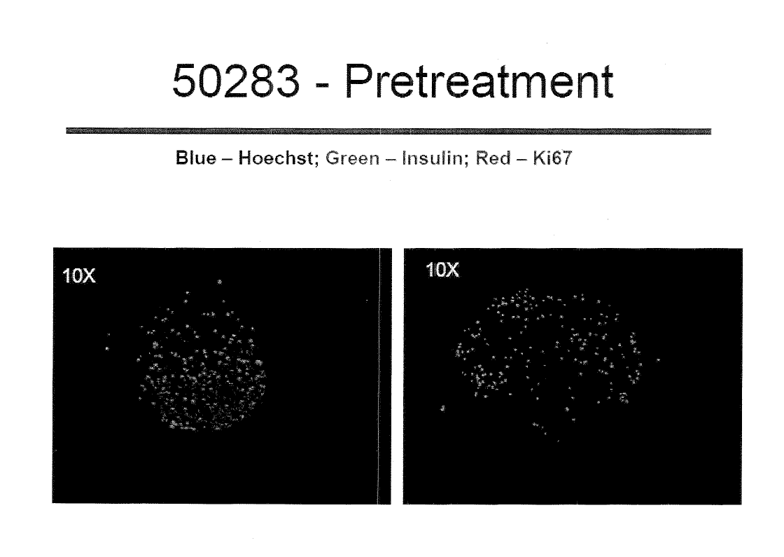

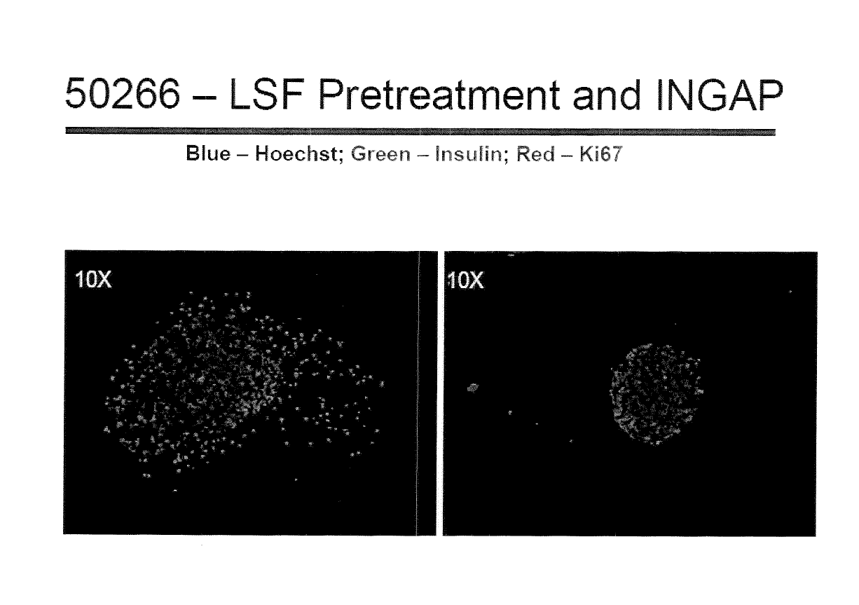

FIG. 8 shows excellent insulin staining and expression of the PDX-1.

In FIGS. 9-14, Hoechst staining (blue) shows DNA, insulin (green) shows insulin, and Ki 67 (red) shows cell cycling or cell proliferation. In particular, FIG. 9 shows staining of an islet of a non-NOD mouse, i.e. a normal islet. FIGS. 10 and 11 show islets from NOD mice that are not yet diabetic, i.e., presumably normal. FIG. 12 is slide from an islet of a saline treated mouse. FIGS. 13 and 14 are slides showing that islets from the LSF pretrement-INGAP group, which stained comparable to controls.

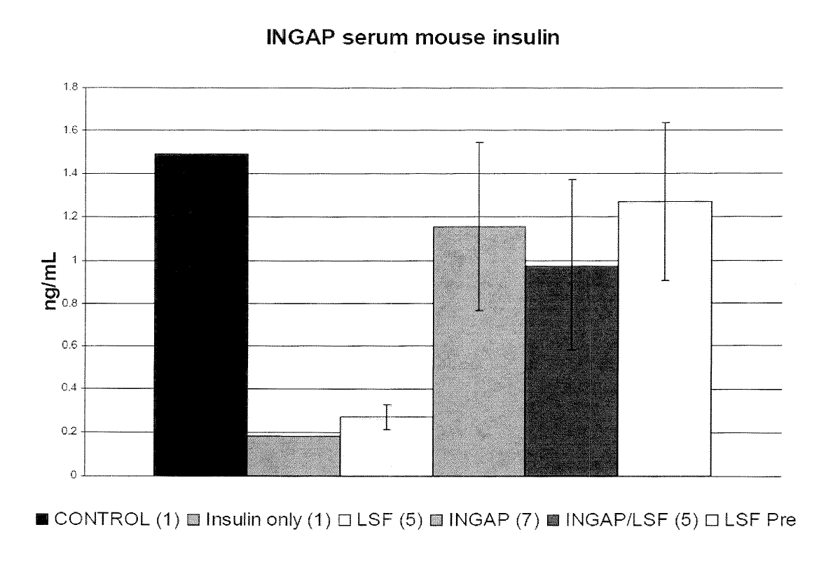

FIG. 15 is a slide showing that all the INGAP treated animals were producing insulin and approximately the same amount. It is only when the combination is used that the insulin was effective to lower BG and only in pre-treatment mice where the average BG level was returning to normal. As can be seen from this and the above data, the remission rate with pre-treatment is unexpected and superior to protocols without such pre-treatment. The overall remission rate is almost two times higher in the pretreatment group than the concurrent or concomitant group. It is two times higher in those animals with severe diabetes, i.e., starting BG level of over 350. The decrease in insulin usage during the treatment period is also unexpected and superior. Finally, the quality of the islets being regenerated is superior in terms of maturation and morphology. There is no remission with LSF alone.

In sum, the combination of a biological response modulator (e.g., LSF) and a beta cell growth or differentiating factor (e.g., INGAP or Ex-4) following pre-treatment with an immune modulator (e.g., LSF) is an effective therapy for T1DM or a disease or condition resulting from the loss of pancreatic islet cells in a patient.

Although the invention has been described in detail with reference to specific embodiments, those of skill in the art will recognize that modifications and improvements are within the scope and spirit of the invention, as set forth in the claims which follow. All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the invention.

1. A method for treating diabetes in a mammal, comprising a first step of administering to the mammal a therapeutically-effective amount of LSF in admixture with a pharmaceutically acceptable carrier, diluent, excipient, adjuvant or vehicle followed by a second step of administering a therapeutically effective amount of INGAP in admixture with a pharmaceutically acceptable carrier, diluent, excipient, adjuvant or vehicle.

2. The method of claim 1, wherein the second step, INGAP is concomitantly administered with LSF.

3. A pharmaceutical composition comprising a LSF and INGAP in admixture with a pharmaceutically acceptable carrier, adjuvant or vehicle, wherein the pharmaceutical composition restores β cell mass and function in a mammal in need thereof.
